(19) United States
(12) Patent Application Publication
(10) Pub. No.: US 2026/0306333 A1
(43) Pub. Date: Oct. 8, 2026

(54) DISTRIBUTED AND REDUNDANT CONTROL ARCHITECTURE FOR SURGICAL TABLE

(71) Applicant: American Sterilizer Company, Mentor, OH (US)

(72) Inventors: Shiva K. Madishetty, Mentor, OH (US); James A. Petrucci, Bluffton, SC (US); John M. Kasunich, Mayfield Heights, OH (US)

(73) Assignee: American Sterilizer Company, (US)

(21) Appl. No.: 19/479,005

(22) PCT Filed: May 14, 2024
(86) PCT No.: PCT/US2024/029275

§ 371 Date: Oct. 27, 2025

Related U.S. Application Data
(60) Provisional application No. 63/466,463, filed on May 15, 2023.

(51) Int. Cl. A61G 13/08; A61G 13/12; G16H 40/63

(52) U.S. Cl. CPC A61G 13/08; A61G 13/1225; A61G 13/123; G16H 40/63

(57) ABSTRACT

A surgical table for supporting a patient during a medical procedure includes a redundant controllers, communication networks and electrically-operated actuators for controlling the table. In the event a controller, communication network or electrically-operated actuator fails, a backup technique takes over thus enabling operation of the table.

### CLAIM OF PRIORITY AND CROSS-REFERENCE TO RELATED APPLICATIONS

This application claims priority to U.S. Provisional Patent Application Ser. No. 63/466,463, filed on May 15, 2023, entitled “DISTRIBUTED AND REDUNDANT CONTROL ARCHITECTURE FOR SURGICAL TABLE,” which is incorporated by reference herein in its entirety.

### FIELD OF INVENTION

The present disclosure relates to a surgical table for prone and lateral positioning, and more particularly to an electrically actuated surgical table that includes redundant control architecture.

### BACKGROUND OF THE INVENTION

Surgical tables are not life support critical systems. However, some level of redundancy and fault tolerance is desired so that a malfunction or component failure during a surgical procedure will not interrupt the procedure.

Conventional surgical tables often use a single central controller that accepts commands from a user and, in response to those commands, activates an appropriate hydraulic actuator(s) to move the table. However, a centralized control architecture has many single points of failure that could disable the entire table. In addition, a centralized architecture results in many wires connecting the central control to the individual sections of the table. These wires flex as the table articulates and are potential failure points.

Such conventional surgical tables lack redundancy and thus there are many single failures that can disable the entire table. Further, the large number of wires/hoses from the central controller to individual actuators adds cost, and failure of these wires/hoses is of concern due to table movement.

### SUMMARY OF THE INVENTION

Aspects of the present invention are directed to an electrically-actuated surgical table that includes redundant control architecture. For example, control architecture of a surgical table in accordance with the invention includes at least two controllers communicatively coupled to multiple electrically-operated actuators distributed about the surgical table, each actuator having a local electrically-operated actuator controller. The surgical table may further include multiple user input devices for receiving user commands, and multiple communication paths/busses that communicatively couple the controllers to the actuator controllers and user input devices. In the event that one of the controllers, one of the communication busses or one of the user input devices experience a fault condition or otherwise fails, the other controller, communication bus or user input device can be used to operate the surgical table. The surgical table may also include separate power zones such that a failure in one zone will leave the remaining zones operational.

Advantageously, by distributing the control across the surgical table the amount of wiring required in the surgical table can be significantly reduced. For example, with distributed control the only connections between table sections are power and communication; all the individual actuator and sensor wires remain within the individual section. Distributing the control also allows some key functions to be duplicated on two (or more) different PC boards such that a failure of one board does not disable the surgical table.

According to one aspect of the invention, a surgical table for supporting a patient during a medical procedure includes: a main support; a seat section for supporting a lower torso and pelvis of the patient, the seat section connected to the main support; a back section for supporting an upper torso of the patient, the back section connected to the seat section, wherein the back section is movable relative to the seat section; at least one electrically-operated actuator coupled to the seat section and the back section, the at least one electrically-operated actuator operative to cause relative movement between the seat section and the back section, a primary controller communicatively coupled to the at least one electrically-operated actuator; and a secondary controller communicatively coupled to the at least one electrically-operated actuator, wherein when the primary controller is in a first state the primary controller is configured to control the at least one electrically-operated actuator and the secondary controller is configured to monitor the primary controller, and when the primary controller is in a second state the secondary controller is configured to control the at least one electrically-operated actuator.

In one embodiment, the surgical table includes a leg section for supporting legs of the patient, the leg section connected to the seat section, wherein the leg section is movable relative to the seat section.

In one embodiment, the at least one electrically-operated actuator includes a plurality of electrically-operated actuators, wherein at least one of the plurality of electrically-operated actuators is coupled to the seat section and the leg section, the at least one of the plurality of electrically-operated actuators communicatively coupled to the primary controller and the secondary controller, wherein the primary controller is configured to control the at least one of the plurality of electrically-operated actuators when in the first state, and the secondary controller is configured to control the at least one of the plurality of electrically-operated actuators when the primary controller is in the second state.

In one embodiment, the secondary controller is configured to monitor the primary controller to determine if the primary controller is in the first state or the second state, and upon the secondary controller determining the primary controller is in the second state the secondary controller is configured to take over control of the at least one electrically-operated actuator.

In one embodiment, the surgical table includes: a primary actuator bus coupled to the primary controller, the secondary controller, and the at least one electrically-operated actuator; and a secondary actuator bus coupled to the primary controller, the secondary controller, and the at least one electrically-operated actuator, wherein when the primary actuator bus is in a first state, communications between the primary controller, the secondary controller and the at least one electrically-operated actuator occur over the primary actuator bus, and when the primary actuator bus is in a second state, communications between the primary controller, the secondary controller and the at least one electrically-operated actuator occur over the secondary actuator bus.

In one embodiment, the at least one electrically-operated actuator includes an electric motor and a motor controller operatively coupled to the electric motor.

In one embodiment, the surgical table includes a first user input device communicatively coupled to the primary controller and the secondary controller, the first user input device configured to receive inputs from the user and communicate the received inputs to the primary controller and the secondary controller.

In one embodiment, the surgical table includes a command bus, wherein the first user input device is communicatively coupled to the primary controller and the secondary controller via the command bus.

In one embodiment, the surgical table includes a second user input device communicatively coupled to the secondary controller, the second user input device configured to receive inputs from the user and communicate the received inputs to the secondary controller.

In one embodiment, the surgical table includes a third user input device communicatively coupled to the secondary controller, the third user input device configured to receive inputs from the user and communicate the received inputs to the secondary controller.

In one embodiment, the secondary controller is configured to communicate commands received from the second user input device and the third user input device to the primary controller via the command bus.

In one embodiment, the at least one electrically-operated actuator includes a plurality of electrically-operated actuators, the surgical table further including: a main power source; a control power switch having a control power input terminal and a plurality of control power output terminals, the control power input terminal coupled to the main power source; and a motor power switch having a motor power input terminal and a plurality of motor power output terminals, the motor power input terminal coupled to the main power source, wherein the primary controller and the secondary controller are electrically connected to respective ones of the plurality of control power output terminals, and the plurality of electrically-operated actuators are electrically connected to respective the motor power output terminal.

According to another aspect of the invention, a method for controlling a surgical table configured to support a patient during a medical procedure is provided, where the surgical table includes a main support, a seat section for supporting a lower torso and pelvis of the patient, the seat section connected to the main support, and a back section for supporting an upper torso of the patient, the back section connected to the seat section, where the back section is movable relative to the seat section. The method includes: controlling at least one electrically-operated actuator with a primary controller, the at least one electrically-operated actuator configured to cause relative movement between the seat section and the back section; monitoring, by a secondary controller, an operational status of the primary controller; and upon the secondary controller determining the primary controller is malfunctioning, the secondary controller taking over control of the at least one actuator from the primary controller.

In one embodiment, monitoring by the secondary controller includes: the secondary controller querying the primary controller; and the secondary controller taking over control of the at least one actuator upon the primary controller failing to respond to the query.

In one embodiment, the surgical table includes a primary actuator bus coupled to the primary controller, the secondary controller, and the at least one electrically-operated actuator, and a secondary actuator bus coupled to the primary controller, the secondary controller, and the at least one electrically-operated actuator, the method further including: determining a status of the primary actuator bus; using the primary actuator bus to communicate between the primary controller, the secondary controller and the at least one electrically-operated actuator when the primary actuator bus is in a first state; and using the secondary actuator bus to communicate between the primary controller, the secondary controller and the at least one electrically-operated actuator when the primary actuator bus is in a second state.

In one embodiment, the surgical table includes a first user input device communicatively coupled to the primary controller and the secondary controller via a command bus, the method further includes: the secondary controller monitoring a status of the command bus; and the secondary controller taking over control from the primary controller of the at least one actuator upon the command bus being in a malfunctioning state.

In one embodiment, the method includes using a first user input device communicatively coupled to the primary controller and the secondary controller to receive inputs from the user and communicate the received inputs to the primary controller and the secondary controller.

In one embodiment, using the first user interface includes communicating to the primary and secondary controllers over a command bus communicatively coupled to the primary controller and the secondary controller via the command bus.

In one embodiment, the method includes using a second user input device to receive inputs from the user and communicate the received inputs to the secondary controller.

In one embodiment, the method includes using a third user input device to receive inputs from the user and communicate the received inputs to the secondary controller.

In one embodiment, using the second user input device or the third user input device includes the secondary controller communicating commands received from the second user input device or the third user input device to the primary controller via a command bus.

To the accomplishment of the foregoing and related ends, the invention, then, comprises the features hereinafter fully described and particularly pointed out in the claims. The following description and the annexed drawings set forth in detail certain illustrative embodiments of the invention. These embodiments are indicative, however, of but a few of the various ways in which the principles of the invention may be employed. Other objects, advantages and novel features of the invention will become apparent from the following detailed description of the invention when considered in conjunction with the drawings.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof.

FIGS. 1A and 1B are top and side views of an exemplary surgical table to which aspects of the present invention may be applied.

FIGS. 2A-2C are schematic diagrams of an exemplary control architecture in accordance with an embodiment of the present invention.

FIGS. 3A-3C are command flow diagrams illustrating flow of a user-entered command when the control system of the surgical table in accordance with the invention is operating normally.

FIGS. 3D-3H are command flow diagrams illustrating flow of a user-entered command when the control system of the surgical table in accordance with the invention is operating abnormally.

FIG. 4 is a schematic diagram illustrating an exemplary power distribution for a control system in accordance with the present invention.

FIG. 5 is a flow chart illustrating exemplary steps for determining which controller (primary or secondary) is operational in accordance with the invention.

FIG. 6 is a flow chart illustrating exemplary steps for determining if the primary and secondary actuator busses are operational in accordance with the invention.

FIG. 7 is a flow chart illustrating exemplary steps for determining if an actuator controller or the secondary controller are operational in accordance with the invention.

### DETAILED DESCRIPTION OF PREFERRED EMBODIMENT

Aspects of the present invention will now be described with reference to the drawings, wherein like reference numerals are used to refer to like elements throughout. Such aspects may be embodied in many different forms and should not be construed as limited to the embodiments set forth herein; rather, these embodiments are provided so that this disclosure will be thorough and complete, and will fully convey its scope to those skilled in the art. Further, it will be understood that the figures are not necessarily to scale.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

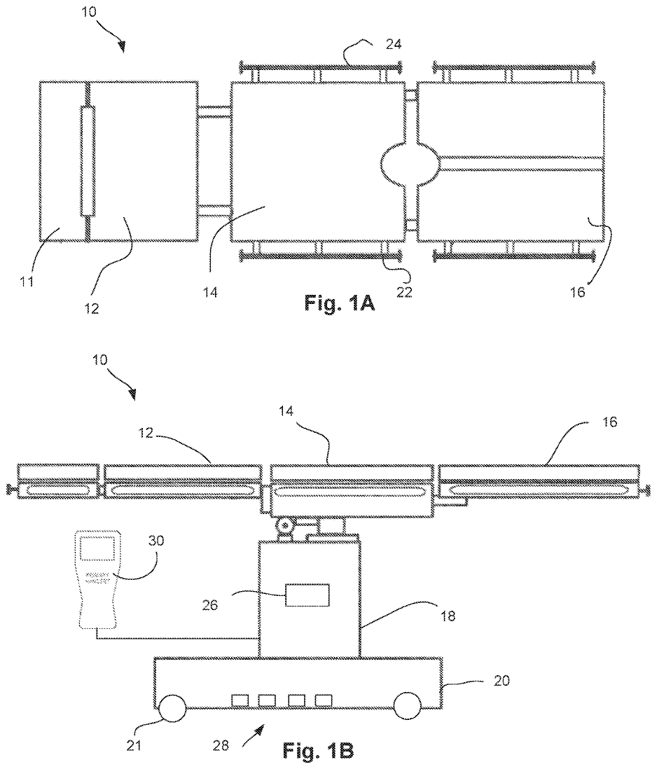

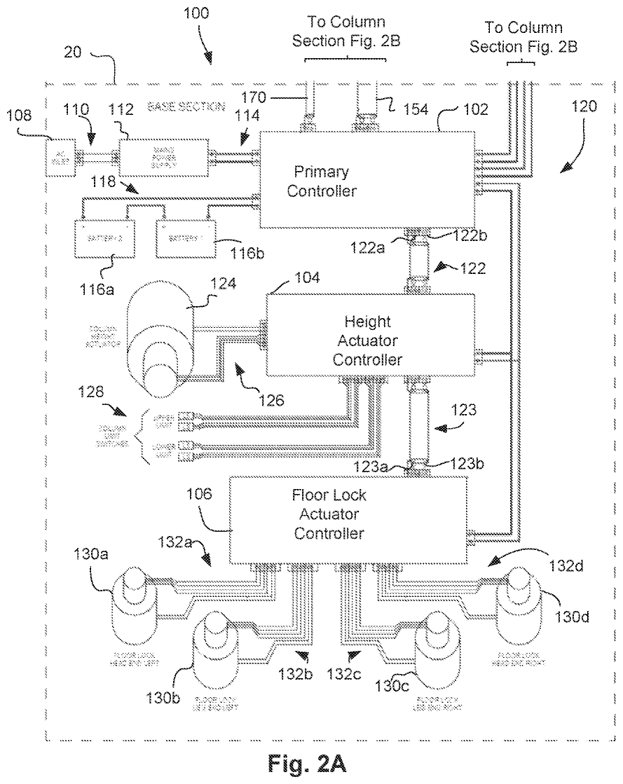

Referring initially to FIGS. 1A and 1B, illustrated are top and side views of an exemplary surgical table 10 to which aspects of the present invention may be applied. The top of the table 10 is formed from three sections, namely, a head section 11 for supporting a patient's head, a back section 12 for supporting a patient's back, a seat section 14 for supporting a patient's lower torso and pelvis, and a leg section 16 for supporting the patient's legs/lower body. The head section 11, back section 12, seat section 14 and leg section 16, which are detachably connectable to each other and can be moved independent of each other to achieve a desired patient position, are connected and assembled on a column section 18 attached to a base section 20 (e.g., a main support in the form of a platform or stand). For example, the seat section 14 may be directly connected to the column section 18, and the head section 11 and leg section 14 may be indirectly connected to the column section 18 through the back section 12 and the seat section 14 respectively. The base section 20 may include wheels 21 that enable the surgical table to be easily moved across a floor. Further, some or all of the surgical table sections may be equipped with side rails 22 24, which can be used for support of the various medical instruments utilized during a surgical procedure.

Arranged on the column 18 is a backup keypad 26, and associated with the column 18 are foot switches 28, while a primary handset 30 is coupled to the table 10. As discussed in further detail below, the backup keypad 26, foot switches 28 and primary handset 30 can be used to control the surgical table 10.

As will be discussed in further detail below, the surgical table 10 includes a plurality of electrically-operated actuator controllers and associated actuators that are operative to move the head section 11, back section 12, body section 14, leg sections 16, column section 18 relative to each other and to lock the wheels in the base section 20 so as to inhibit movement of the base section (and thus the surgical table).

For example, one or more electrically-operated actuators may be coupled between the seat section 14 and the back section 12 or between the seat section 14 and the leg section 16, the one or more electrically-operated actuators operative to cause relative movement between the seat section 14, the back section 12 and/or the leg section 16. Each section may also include electrically-operated actuators that enable movement of one portion of the section relative to another portion of the same section. As discussed in further detail below, a primary controller and a secondary controller are each communicatively coupled to the electrically-operated actuators, the primary controller operative to coordinate operation of the electrically-operated actuators during normal operation. The secondary controller is operative to monitor the primary controller, and in the event the primary controller is deemed inoperative, the secondary controller takes over and coordinates control of the electrically-operated actuators.

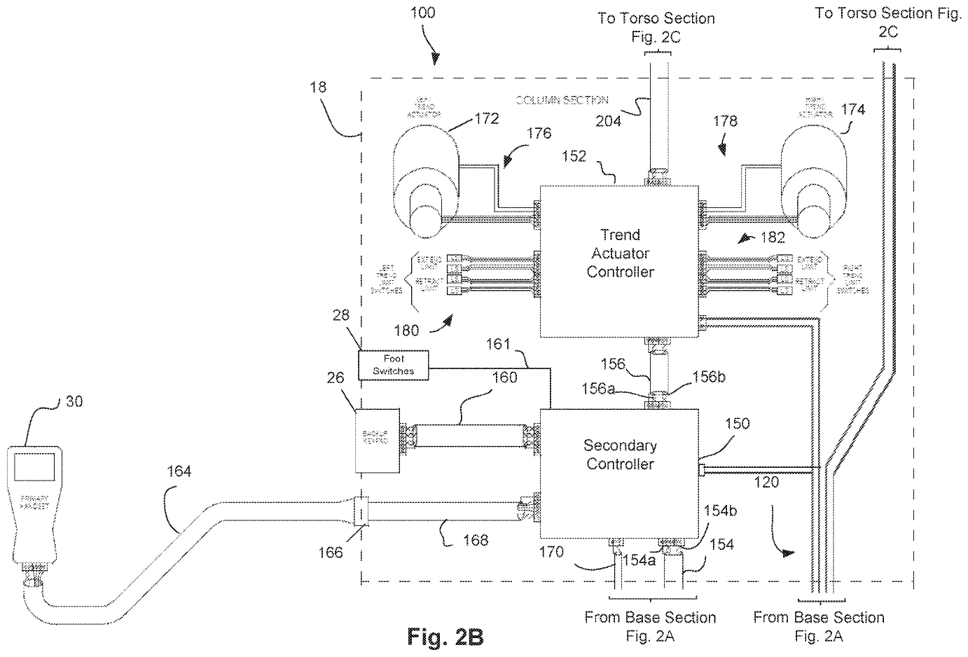

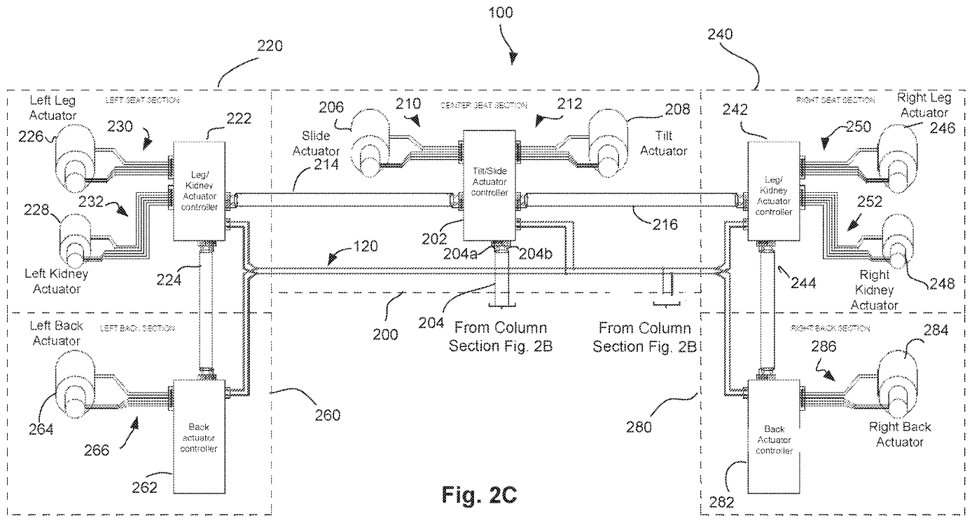

Referring to FIGS. 2A-2C, illustrated are schematic diagrams of an exemplary control architecture 100 for a surgical table 10 in accordance with the present invention. The large, dashed boxes in FIGS. 2A-2C represent the major physical sections of the surgical table 10, where each section is movable relative to the adjacent sections and any wiring crossing the dashed lines is subject to flexing. These sections include the column section 18, the base section 20, the center seat section 200, the left seat section 220, the right seat section 240, the left back section 260 and the right back section 280. It is noted that while certain controllers are shown in a specific location of the surgical table, this is merely exemplary and such controllers could be moved to other sections without departing from the scope of the invention.

Beginning with the base section 20 as shown in FIG. 2A, a primary controller 102, a height actuator controller 104 and a floor lock actuator controller 106 are each mounted on the base section 20. Power, such as 120 VAC or 240 VAC, is provided to the base section via A/C inlet 108 and power conductor 110 electrically connects the A/C inlet 108 to a power supply 112, which generates power used by various components of the surgical table. Power generated by the power supply 112 is provided to the primary controller 102 via conductors 114. Further, secondary power in the form of batteries 116a, 116b also provides power to the primary controller 102 via secondary conductors 118. A power distribution circuit (not shown) of the primary controller 102 selectively switches between the A/C power and the battery secondary power. Normally, the power distribution circuit will select the power provided by the power supply 112, and upon such power being lost the circuit will switch to the battery power. The selected power is provided to the other components of the system via power bus 120.

The primary controller 102 communicates with the height actuator controller 104 and the floor lock actuator controller 106 via communication bus 122, 123, such as a CAN bus or the like, to provide supervisory control of the respective sections. The communication bus 122, 123 may include an outer shield with two sets of twisted-pair conductors arranged within the shield, where a first pair of conductors forms part of a first (e.g., primary) actuator communication bus 122a, 123a and a second pair of conductors forms part of a second (e.g. backup) actuator communication bus 122b, 123b. During normal operation, communications are exchanged over the primary actuator communication bus 122a, 123a. However, if the primary actuator communication bus 122a, 123a fails, communication then are performed over the second actuator communication bus 122b, 123b. As discussed in more detail below, the primary controller 102 (and a secondary controller) oversee operations of the various sections of the surgical table 10 to provide a desired orientation of each section of the table 10 relative to other sections of the table.

The height actuator controller 104 is electrically connected to a column height actuator 124, such as an electric motor, via electrical conductors 126. The electrical conductors 126 may include power conductors for driving the column height actuator 124, and feedback conductors for obtaining data corresponding to motion of the column height actuator 124. Further, limit switches 128 corresponding to height limits of the column section 18 (e.g., upper and lower height limit switches) are provided to inputs of the height actuator controller 104. The height actuator controller 104 is operative to receive control commands provided by the primary controller 102 (or as discussed in further detail below commands provided by a secondary controller), and drive the column height actuator 124 to alter a height of the column section 18 (e.g., raise or lower the column height). In the event a limit switch is actuated, the height actuator controller 104 will stop movement of the column section 18 for a direction that corresponds to the actuated limit switch. Operation of the height actuator 124 in the opposite direction (i.e., the direction in which the limit switch is not actuated) is still permitted.

The floor lock actuator controller 106 is electrically connected to a plurality of floor lock actuators to enable or inhibit motion of the base section 20 (and thus the entire surgical table) relative to the floor. In the illustrated embodiment four actuators are electrically connected to the floor lock actuator controller 106. Specifically, a head end left side floor lock actuator 130a, a leg end left side floor lock actuator 130b, a leg end right side floor lock actuator 130c, a head end right side floor lock actuator 130d are connected to the floor lock actuator controller 106 via respective electrical conductors 132a, 132b, 132c, 132d (the conductors including power conductors and feedback conductors as discussed above). To inhibit motion of the base section 20 relative to the floor, the floor lock actuator controller 106, under the supervision of the primary or secondary controller, receives a command to extend the floor locks. To enable motion of the base section 20 relative to the floor, the floor lock actuator controller receives a command to retract the floor locks. In response to the received commands, the floor lock actuator controller 106 drives the respective actuators 130a-130d to either the extend position (which causes the base section 20 to be lifted upward such that the wheels 21 no longer contact the floor) or the retract position (which causes the base section 20 to be lowered back to the floor such that the wheels 21 contact the floor).

With additional reference to FIG. 2B, illustrated is the control architecture for the column section 18. The column section 18 includes the backup (secondary controller) 150 and a trend actuator controller 152, the secondary controller 150 and trend actuator controller 152 receiving power from the power bus 120. The secondary controller 150 and trend actuator controller 152 are communicatively coupled to the primary controller 102 of the base section 20 via bus 154, 156. Like the bus 122, 123 of the base section 20, the bus 154, 156 includes a primary bus 154a, 156a and a secondary (backup) bus 154b, 156b, which in the illustrated embodiment are each formed by a twisted pair conductor, both twisted pair conductors being disposed within a common shield. The secondary controller 150 is communicatively coupled to a backup keypad 26 and foot switches 28 via first communication cable 160 and electrical conductors 161. The backup keypad 26, which may be mounted on a portion of the column section 18, provides one technique for a user to input commands for controlling the surgical table 10, while the foot switches 28, which may be attached to the column section 18, provide another technique for a user to input commands for controlling the surgical table. In other exemplary embodiments, the foot switches 28 may be a separate assembly that are in contact with a least a portion of the floor and may be movable at a user's discretion. The secondary controller 150 is also communicatively coupled to a primary handset 30 via a second communication cable 164, which is detachably couplable to connector 166. Shielded conductors 168 communicatively couple the secondary controller 150 to the connector 166 (and thus to the primary handset 30). The primary handset 30 provides another technique for a user to input commands for controlling the surgical table 10. Notably, the primary handset 30 is also communicatively coupled to the primary controller 102 through the command bus 170 (which includes the shielded conductors 164, 168), which may be a shielded twisted pair conductor that communicatively couples the secondary controller 150 to the primary controller 102.

The trend actuator controller 152 is electrically connected to trend actuators, e.g., left trend actuator 172 and right trend actuator 174 via electrical conductors 176, 178, respectively. Like the other conductors that connect a controller to an actuator, the electrical conductors 176, 178 may include power conductors for driving the trend actuators 172, 174, and feedback conductors for obtaining data corresponding to motion of the trend actuators 172, 174. Further, limit switches 180, 182 corresponding to trend limits of the trend function (e.g., upper and lower height limit switches for each trend actuator) are provided to inputs of the trend actuator controller 152. The trend actuator controller 152 is operative to receive control commands provided by the primary controller 102 or the secondary controller 150 and drive the left and right trend actuators 172, 174 to alter a height of one end of the patient table relative to an opposite end of the patient table. In the event a limit switch is actuated, the trend actuator controller 152 will stop movement of the trend actuator 172, 174 for a direction that corresponds to the actuated limit switch. Motion corresponding to the non-actuated limit switch is still permitted.

Referring now to FIG. 2C, illustrated is the control architecture of the center seat section 200, the left seat section 220, the right seat section 240, the left back section 260 and the right back section 280, each of which receive power from the column section 18 via power bus 120. The center seat section 200 includes a tilt/slide actuator controller 202 that is communicatively coupled to the trend actuator controller 152 of the column section 20 via bus 204. Like the other communication busses, bus 204 includes a primary bus 204a and a secondary (backup) bus 204b, which in the illustrated embodiment are each formed by a twisted pair conductor both disposed within a common shield. The tilt/slide actuator controller 202 is electrically connected to a slide actuator 206 and a tilt actuator 208 via electrical conductors 210, 212, respectively. Like the other actuator conductors, the conductors 210, 212 may include power conductors for driving the slide and tilt actuators 206, 208, and feedback conductors for obtaining data corresponding to motion of the slide and tilt actuators 206, 208. The slide/tilt actuator controller 202 is operative to receive control commands provided by the primary controller 102 or the secondary controller 150 and drive the slide and tilt actuators 206, 208 to transversely move the center seat section and/or tilt the center seat section.

The tilt/slide actuator controller 202 is communicatively coupled to leg/kidney actuator controllers 222, 242 of the left and right seat sections 220, 240, via communication bus 214 and 216, respectively. Further, the leg/kidney actuator controllers 222, 242 are communicatively coupled to back actuator controllers 262, 282 of the left and right back sections 260, 280 via communication bus 224, 244, respectively. Each bus 214, 216, 224, 244 includes primary and secondary buses 214a, 214b, 216a, 216b, 224a, 224b and 244a, 244b, respectively, which may be formed as twisted pair conductors within a common shield. Accordingly, the primary controller 102, secondary controller 150 and each actuator controller can communicate with one another over the bus connections 122, 123, 154, 156, 204, 214, 216, 224 and 244, which are collectively referred to as the “redundant actuator bus”, where bus connections 122a, 123a, 154a, 156a, 204a, 214a, 216a, 224a and 244a of the redundant actuator bus are collectively referred to as the “primary actuator bus” and bus connections 122b, 123b, 154b, 156b, 204b, 214b, 216ab 224b and 244b of the redundant actuator bus are collectively referred to as the “secondary actuator bus”.

Moving to the left and right seat sections 220, 240, each section includes the aforementioned leg/kidney actuator controllers 222, 242, which receive power from the power bus 120. The left seat section leg/kidney actuator controller 222 is electrically connected to a left leg actuator 226 and a left kidney actuator 228 via electrical conductors 230, 232, respectively, while the right seat section leg/kidney actuator 242 is electrically connected to right leg actuator 246 and right kidney actuator 248 via conductors 250, 252, respectively. The electrical conductors 230, 232, 250, 252 may include power conductors for driving the actuators and feedback conductors for obtaining data corresponding to motion of the actuators as previously discussed.

Lastly, the left and right back sections 260, 280 include the aforementioned back actuator controllers 262, 282, which also receive power from the power bus 120. The left back section back actuator controller 262 is electrically connected to a left back actuator 264 via electrical conductors 266 (power and feedback conductors), while the right back section back actuator controller 282 is electrically connected to right back actuator 284 via conductors 286 (power and feedback conductors).

In view of FIGS. 2A-2C, redundant operation of the system will now be discussed. There are several possible scenarios for component failure, including failure of an actuator controller, failure of the primary controller, failure of the primary actuator bus, and failure of the command bus. As discussed below, a variety of methods may be used to detect hardware failures and allow transition to one of the backup techniques.

Addressing first the detection of a failed actuator controller, both the redundant actuator bus and the command bus use a principal/agent architecture in which the principle sends out a message at a specific time interval (for example, every 50 milliseconds). Each agent can be assigned a time slot, measured from the end of the principle message, and may be expected to reply during that time slot. For example, agent 1 may be expected to send its reply 4 ms after the principle message ends, agent 2 sends its reply 7 ms after the principle message, and each subsequent agent sends its message 3 ms later. Thus, agent 8 would send its message 25 ms after the principle message ends. Since each agent is expected to respond during a specific time slot, the absence of a response during that time slot can be used to infer that a specific agent (e.g., an actuator controller) has failed. Failure of a specific actuator controller can be reported to indicate the table requires repair, and motions associated with the failed controller are disabled. Other table motions will still be available.

With respect to the primary and secondary controllers, under normal conditions the primary controller 102 operates as the principle and the secondary controller 150 is an agent on the bus (and therefore responds in its respective time slot). However, since the secondary controller 150 can see the principle message and all replies, the secondary controller 150 can detect if the primary controller 102 has stopped sending messages and, if so, take over for the primary controller 102. For example, during a normal (first) state of operation, i.e., when no faults are present and the primary controller 102 is operating normally, the primary controller 102 controls the actuator controllers 104, 106, 152, 202, 222, 242, 262, 282 by sending and receiving messages to/from these controllers over the redundant actuator bus. In this regard, the primary controller 102 may send commands over the primary actuator bus to one or more of the actuator controllers to operate its actuators in a particular manner, e.g., forward actuator motion, reverse actuator motion, fast/slow speed, target positions for the actuator, coordinated motion of multiple actuators, etc. Such commands may be based on user input from the primary handset 30, backup keypad 26 and/or foot switches 28. Further, the secondary controller 150 is operative to monitor the messages sent by the primary controller 102. So long as messages are sent by the primary controller 102 in an expected format and time slot, the secondary controller 150 may conclude the primary controller 102 is operating normally and take no action other than to continue such monitoring. However, if the messages from the primary controller 102 cease or are not in the expected time window/format, the secondary controller 150 may conclude the primary controller 102 is operating abnormally and therefore take over control of the system.

In the case of the redundant actuator bus, the table controller (i.e., the primary controller 102 or the secondary controller 150, depending on which is acting as the principle) monitors the health of both busses. When the surgical table 10 is idle, the table controller alternates between the two busses to confirm both are operational. For example, the table controller can send messages on both the primary and secondary actuator busses. After 100 ms, the table controller will have sent messages on both busses and should have received replies from all actuator controllers on both busses. If the table controller gets replies on one bus but not the other, then the bus in which no replies were received is reported as failed and all communication is directed over the other bus.

For the user input devices, the primary handset 30 is connected to the command bus 168, 170 with its own control unit and operates as an agent on the command bus. The backup keypad 26 and foot switches 28 do not have their own controller. Instead, the backup keypad and the foot switches are directly connected to the secondary controller 150, which forwards their commands to the primary controller 102 during its time slot on the command bus. If the command bus fails, the primary controller 102 can no longer receive any user commands. However, the secondary controller 150 will detect the loss of the command bus and take over control of the system. In this scenario, the secondary controller 150 also cannot receive commands from the primary handset 30 as the command bus is down, but the table 10 can still be operated from the backup keypad 26 and/or foot switches 28.

If the secondary controller 150 fails, the primary controller 102 detects such failure because the secondary controller 150 is no longer replying to messages on either bus. The backup keypad 26 and foot switches 28 will no longer work as these devices require the secondary controller 150 to communicate to the primary controller 102. However, the surgical table 10 can still be operated from the primary handset 30 and primary controller 102.

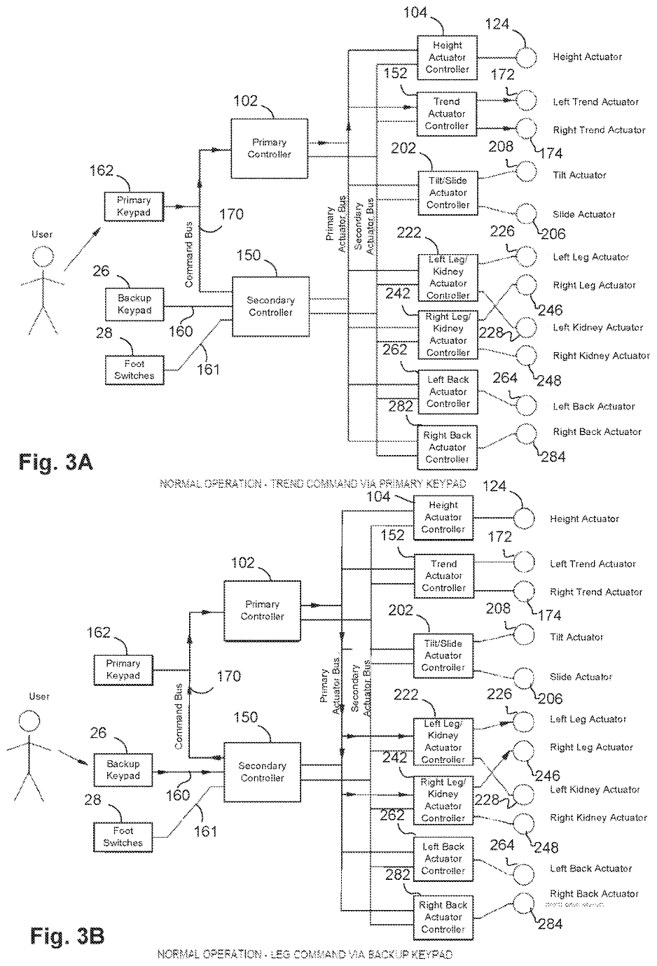

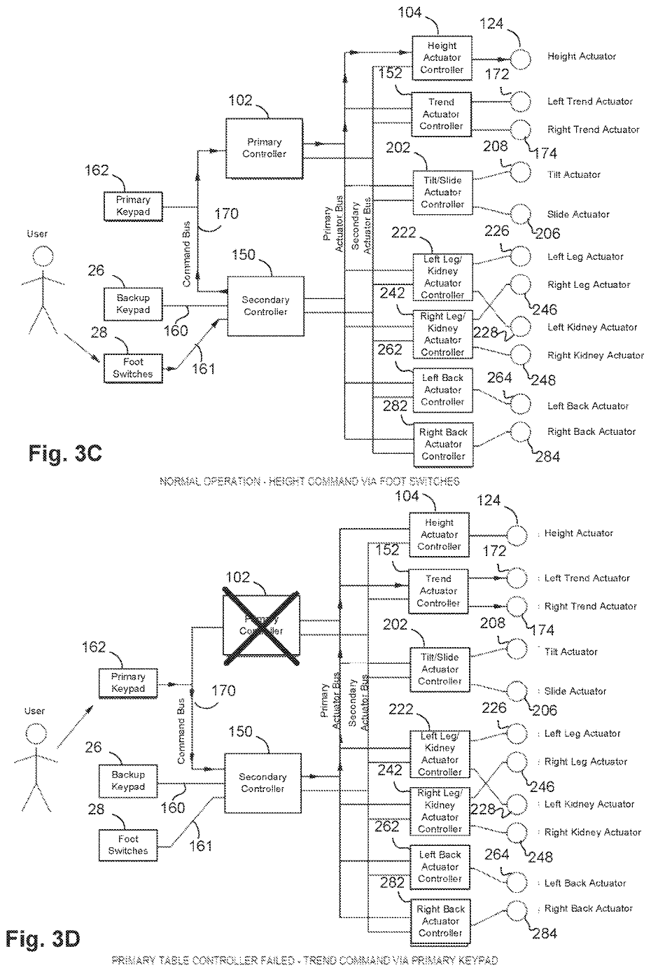

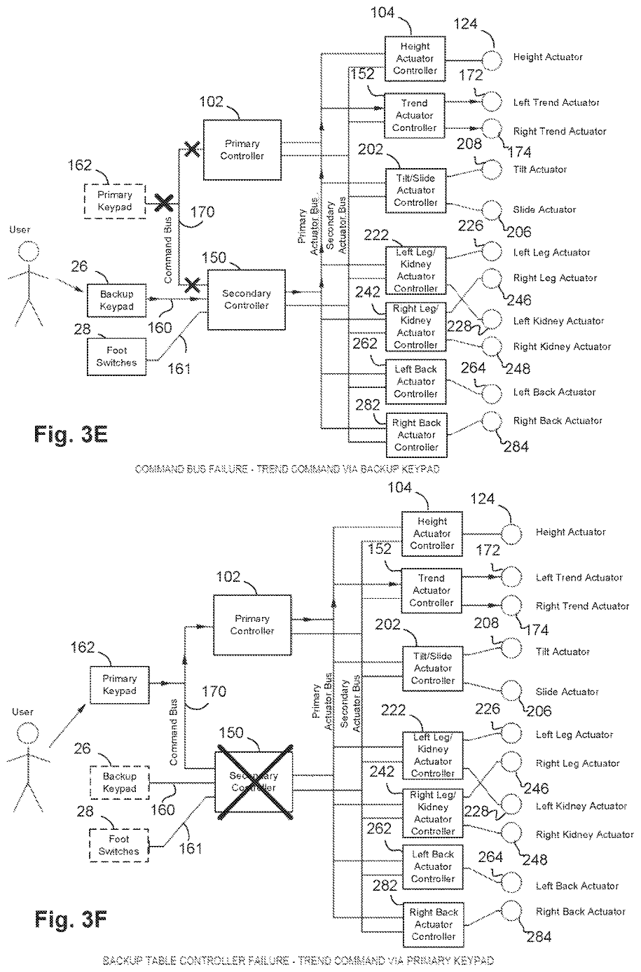

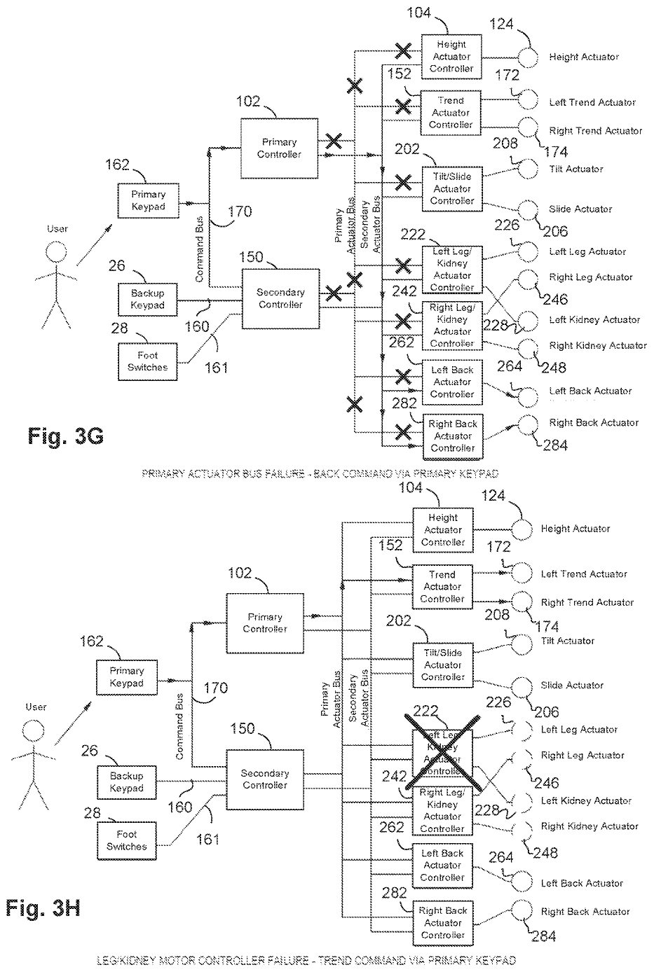

Referring now to FIGS. 3A-3H, command flow diagrams are provided that illustrate operation of a surgical table in accordance with the invention. FIGS. 3A-3C illustrate normal operation of the surgical table with the flow of data/commands from the user to the actuators shown with arrows, while FIGS. 3D-3H illustrate alternate paths for commands when one component has failed or is otherwise not working properly the (inoperable/failed component marked with a bold X).

Beginning with FIG. 3A, illustrated is command flow diagram for normal operation of a trend command entered through the primary keypad 30. More particularly, a user, through the primary keypad 30, selects a trend command (e.g., via a button or graphic input) and the primary keypad 30 communicates the trend command to the primary controller 102 over the command bus 170. The primary controller 102, via the primary actuator bus, instructs the trend actuator controller 152 to operate the left and right trend actuators 172, 174 to produce the desired table movement.

FIG. 3B is a command flow diagram illustrating normal operation of a leg command via the backup keypad. A user enters the desired leg command into the backup keypad 26, which communicates the command to the secondary controller 150 over conductors 160. The secondary controller 150, knowing the primary controller 102 is operating normally, forwards the leg command to the primary controller 102 over the command bus 170. The primary controller 102, via the primary actuator bus, instructs the left and/or right leg/kidney actuator controllers 222, 242 to operate the left and/or right leg actuators 226, 246 to produce a desired table movement.

FIG. 3C is a command flow diagram illustrating normal operation of a height command via the foot switches. A user instructs the system to change the height of the table using the foot switches 28, and such user input is communicated to the secondary controller 150 over conductors 161. The secondary controller 150, knowing the primary controller 102 is operating normally, forwards the height command to the primary controller 102 over the command bus 170. The primary controller 102, via the primary actuator bus, instructs the height actuator controller 104 to operate the height actuator 124 to produce a desired table motion.

Moving now to FIGS. 3D-3H, these figures illustrate command flow diagrams when one component of the system is not operating normally. For example, FIG. 3D is a command flow diagram showing a trend command when the primary controller 102 has failed. In the illustrated example, a user enters the trend command into the primary keypad 30. Normally, this command would be routed to the primary controller 102. However, the secondary controller 150, by monitoring messages on the actuator bus, has determined that the primary controller 102 is faulty and therefore has taken over control of the system. The secondary controller 150 receives the trend command from the primary keypad 30 over the command bus 170 and, via the primary actuator bus, instructs the trend actuator controller 152 to operate the left and/or right trend actuators 172, 174 to produces a desired table movement. Accordingly, although the primary controller 102 is inoperative, from the user's perspective the fault is transparent, and the table may be operated normally.

Moving to FIG. 3E, illustrated is a command flow diagram for a trend command via the backup keypad when the command bus 170 has failed. In the example of FIG. 3E, since the command bus 170 is down user entries cannot be made via the primary keypad 30, as it has no way to communicate to the primary or secondary controllers. However, the table can still be controlled via the backup keypad 26 (or foot switches 28, if desired). In this regard, a user enters the trend command into the backup keypad 26 and the command is routed to the secondary controller 150 via the conductors 160. Normally, the secondary controller 150 would then route the trend command to the primary controller 102. However, since the command bus 170 is down the secondary controller 150, after receiving the trend command via the backup controller, determines (or has previously determined) communication via the command bus 170 to the primary controller 102 is not possible. Therefore, the secondary controller 150 takes over control of the table and, via the primary actuator bus, instructs the trend actuator controller 152 to operate the left and/or right trend actuators 172, 174 to produces a desired table movement.

FIG. 3F illustrates a command flow diagram in which a trend command is issued via the primary keypad while the secondary controller 152 has failed. In the example of FIG. 3F, since the secondary controller 150 has failed, user control via the backup keypad 26 and the foot switches 28 is not possible. However, the table can still be controlled via the primary keypad 30. In this regard, a user enters the trend command into the primary keypad 30 and the command is routed to the primary controller 102 via the command bus 170. The primary controller 102, via the primary actuator bus, instructs the trend actuator controller 152 to operate the left and/or right trend actuators 172, 174 to produces a desired table movement.

Moving to FIG. 3G, illustrated is a command flow diagram for a back command via the primary keypad when the primary actuator buss has failed. A user enters the back command into the primary keypad 30, which communicates the command to the primary controller 102. The primary controller, which is operating normally and as the principle, detects (or has already detected) that the primary actuator bus has failed and therefore uses the secondary actuator bus to instruct the left back actuator controller 262 and the right back actuator controller 282 to operate the left and/or right back actuators 264, 284, respectively, to produces a desired table movement. Accordingly, although the primary actuator bus is inoperative, from the user's perspective the fault is transparent, and the table may be operated normally.

Finally, FIG. 3H illustrates a command flow diagram for a trend command while the left leg/kidney actuator controller 222 is inoperative. A user enters the trend command via the primary keypad 30 and the command is communicated to the primary controller 102 via the command bus 170. Although not shown, it is noted the user may also enter the trend command in the backup keypad 26. The primary controller 102, via the primary actuator bus, instructs the trend actuator controller 152 to operate the left and/or right trend actuators 172, 174 to produces a desired table movement. Since the left leg/kidney actuator controller 222 is not needed for the trend command, there is no issue with performing the trend command. In other words, all other table functions are also possible so long as they do not involve the left leg/kidney actuator 226, 228. It is noted, however, that since the left leg/kidney actuator controller 222 has failed, any motion associated with this controller is inhibited. This includes motion of the right leg/kidney actuators 246, 248, which technically could operate as the right leg/kidney actuator controller 242 is operable. Inhibiting the right-side leg/kidney actuators from moving is necessary as the leg/kidney actuators are coordinated with each other and thus both sides are inhibited.

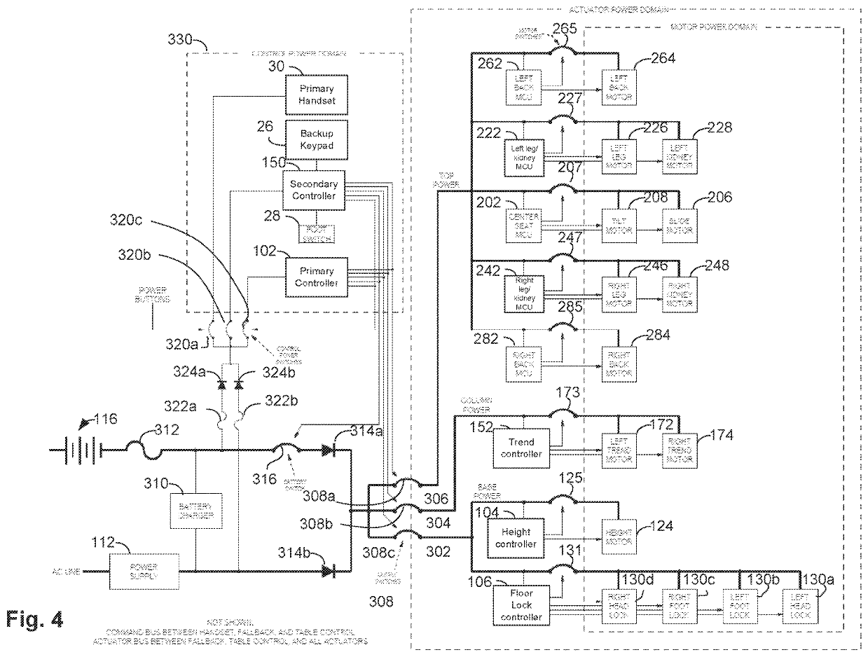

Moving now to FIG. 4, illustrated is a power distribution schematic diagram for the surgical table in accordance with the invention. In the illustrated embodiment, the table is divided into three power zones. The first zone 302 contains the floor lock and height actuator controllers and actuators, the second zone contains the trend actuator controller and actuators, and the third zone contains all the tabletop actuator controllers and actuators (tilt, slide, leg, kidney, and back). The three zones are fed through electronic circuit breakers 308 located on the table controller; these breakers will trip if there is an overload or short circuit in one zone, keeping the other zones working.

With continued reference to FIG. 4, AC power is provided to power supply 112, which converts the AC power to DC at a desired voltage. The power supply 112 powers a battery charger 310, which is electrically connected to battery 116 through fuse 312 and maintains the battery at the proper charge when the table is connected to AC power. The power supplied by the battery 116 as well as the power provided by the power supply 112 are provided to circuit breakers 308 through blocking diodes 314a, 314b, respectively. Electrically in series between the fuse 312 and the diode 314a is a battery switch 316, which enables the battery to be disconnected from breakers 308, if desired. As discussed in more detail below, circuit breakers 308 feed the various actuator power zones.

The power supplied by the battery 116 and the power provided by the power supply 112 are also provided to switches 320s, 320b, 320c through respective fuses 322a, 322b and diodes 324a, 324b respectively. The switches 320a, 320b, 320c may be in the form of circuit breakers, power switches or the like, and may be controlled via user-operable buttons (not shown). The switches 320a, 320b, 320c provide power to the control power domain section 330 of the surgical table. Specifically, switch 320a provides power to the primary handset, switch 320b provides power to the secondary controller 150, the backup keypad 26 and the foot switches 28, and switch 320c provides power to the primary controller 102.

Referring back to circuit breakers 308, top power circuit breaker 308a provides power to the top of the surgical table (left and right back actuator controllers 262, 282 and actuators 264, 284, left and right leg/kidney actuator controllers 222, 242 and actuators 226, 228, 246, 248, and the tilt/slide actuator controller 202 and actuators 206, 208). Column power circuit breaker 308b provides power to the column of the surgical table, namely the trend actuator controller 152 and actuators 172, 174, and base power circuit breaker 308c provides power to the base section, namely, the height actuator controller 104 and actuator 124, and the floor lock actuator controller 106 and actuators 130a-130d.

Further, each actuator (or group of related actuators) includes a respective circuit breaker to connect/disconnect the actuator(s) from the main circuit breaker 308. Specifically, circuit breakers 265, 227, 207, 247 and 285 electrically connect the left back actuator 264, the left and right leg actuators 226, 246, the tilt and slide actuators 206, 208, the left and right kidney actuators 228, 248 and the left and right back actuators 264, 284, respectively, to the top power circuit breaker 308a. Similarly, circuit breaker 173 electrically connects/disconnects the left and right trend actuators 172, 174 from column power circuit breaker 308b. Similarly, circuit breaker 125 electrically connects/disconnects the column height actuator 124 from base power circuit breaker 308c, while circuit breaker 131 electrically connects/disconnects the floor lock actuators 130a-130d from base power circuit breaker 308c.

By dividing the power distribution of the table as shown in FIG. 4, certain sections of the table can operate even when one or more actuator controllers or actuators are down and disconnected from power. Further, the power distribution of FIG. 4 allows the table to operate when either the primary controller 102 or the secondary controller 150 is down and disconnected from power.

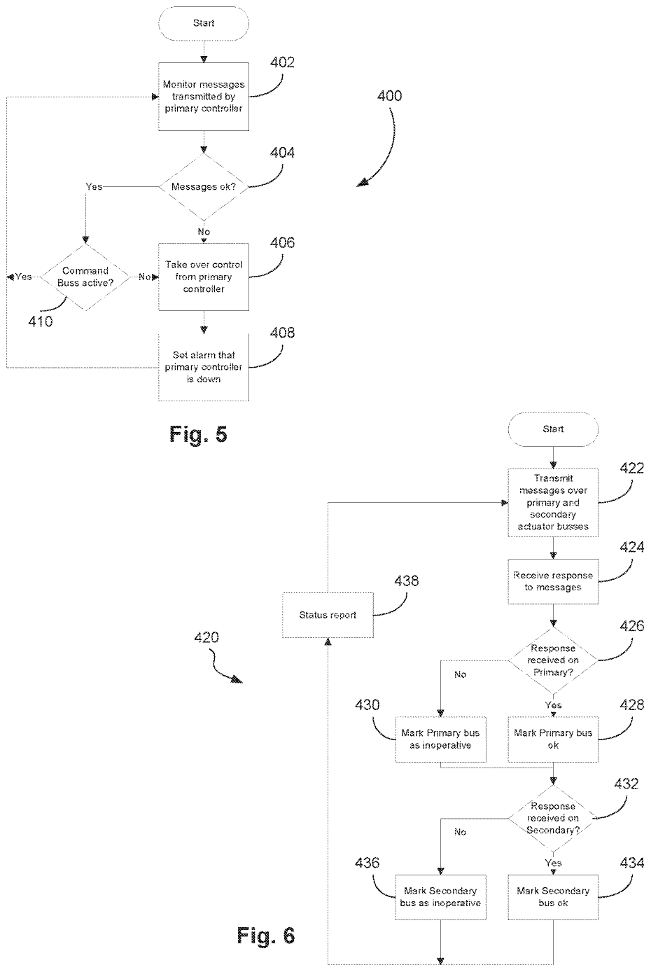

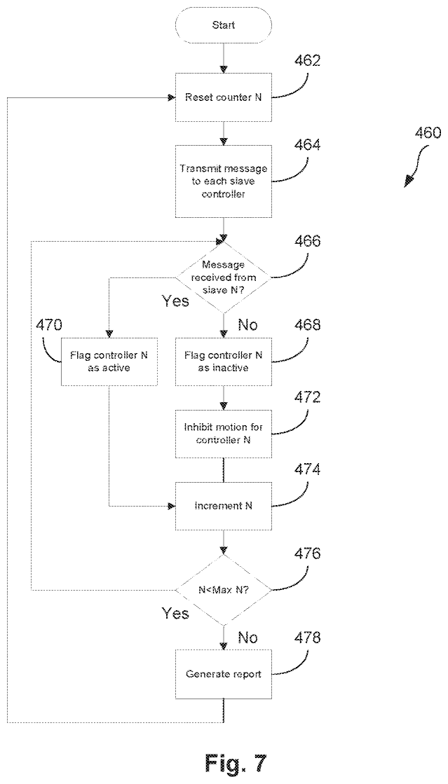

Referring now to FIGS. 5-7, illustrated are flow charts for monitoring the surgical table control system in accordance with the invention. FIG. 5 illustrates exemplary steps for determining if the primary controller or the secondary controller is the active (principle) controller, FIG. 6 illustrates exemplary steps for determining if the primary and secondary actuator busses are active, and FIG. 7 illustrates exemplary steps for determining if the agent controllers are active on the communication network. The steps illustrated in FIGS. 5-7 may be executed by the primary controller 102 and/or the secondary controller 150 as discussed in further detail below.

Starting with FIG. 5, illustrated are exemplary steps of a method 400 for determining if the secondary controller 150 should take over control of the table 10 from the primary controller 102, where the method is executed by the secondary controller 150. Beginning at step 402, the secondary controller 150 monitors the messages on the actuator bus to see if the primary controller 102 is transmitting messages (since the secondary controller 150 is an agent on the actuator bus the secondary controller 150 can see all messages on the bus, including those from the primary controller 102). At step 404 the secondary controller 150 determines if messages are present on the actuator bus. If messages are not present on the actuator bus, then the primary controller 102 is deemed to be inoperative and the method moves to step 406 where the secondary controller 150 takes control of the table 10 from the primary controller 102. At step 408 the secondary controller 150 sets a flag/alarm to indicate to the appropriate personnel that the table requires attention, and the method then stops and the secondary controller 150 remains the principle controller until the table 10 is serviced. Alternatively, the secondary controller 150, while acting as the principle controller, may continue to monitor the actuator bus for messages sent by the primary controller 102 and, if detected, transfer control back to the primary controller 102. In other words, instead of the method stopping at step 408 it may move back to step 402 and repeat.

Moving back to step 404, if messages are present on the actuator bus, then it may be concluded that the primary controller 102 is operative. The method then moves to step 410 where the secondary controller 150 determines if the command bus 170 is active. For example, the secondary controller 150 may periodically query the primary controller 102 over the command bus 170 and wait for a response. Additionally or alternatively, the secondary controller 150 may monitor messages provided by the primary keypad 162. If a response to the query is received by the secondary controller 150 via the command bus 170 and/or messages are detected from the keypad 162 on the command bus 170, then it may be concluded the command bus 170 is operational and table control remains with the primary controller 102. However, if a response to the query is not received by the secondary controller 150 over the command bus 170 and/or no messages are detected from the keypad 162 on the command bus 170 over a predetermined time period, the secondary controller 150 concludes the command bus 170 is inoperative and takes control of the table 10 as indicated at steps 406 and 408 as discussed above. The method then stops and the secondary controller 150 remains the principle controller until the table is serviced. Alternatively, the secondary controller 150, while acting as the principle controller, may continue to query the primary controller 102 over the command bus and/or monitor for messages from the keypad 162 on the command bus 170 and, if a response or messages are received/detected, transfer control back to the primary controller 102. In other words, instead of the method stopping at step 408 it moves back to step 402 and repeats.

Moving to FIG. 6, illustrated are exemplary steps of a method 420 for determining if the primary and secondary actuator busses are operational. In contrast to the method of FIG. 5, the method of FIG. 6 may be executed by the active principle controller (i.e., the controller that is controlling the table 10) and thus either the primary controller 102 or the secondary controller 150 may execute the method of FIG. 6. Beginning at step 422, the active principle controller transmits a message over both the primary and secondary actuator busses. Such message may be transmitted, for example, during a period in which the table 10 is idle. Next at step 424, and in response to the messages transmitted at step 422, the active controller attempts to receive messages over each of the primary actuator bus and the secondary actuator bus. At step 426 the active controller checks if a response was actually received on the primary actuator bus and if so, the method moves to step 428 where the primary actuator bus is marked as operational, and the method moves to step 432. However, if a response is not received on the primary actuator bus, then the method moves to step 430 where the primary actuator bus is marked as inoperative, and the method moves to step 432 to check the secondary actuator bus.

At step 432 the active principle controller checks if a response was actually received on the secondary actuator bus. If a response is received on the secondary actuator bus, then at step 434 the secondary actuator bus is marked as operational, and the method moves to step 438 as discussed below. However, if a response is not received on the secondary actuator bus, then the method moves to step 436 where the secondary actuator bus is marked as inoperative. The method moves to step 438 where a status report for the actuator bus is generated. Such status report may be used to flag personnel that the table 10 requires maintenance/repair. The method then moves back to step 422 and repeats.

Moving to FIG. 7, illustrated are exemplary steps of a method 460 for determining if any of the actuator controllers are operational. Like the method of FIG. 6, the method of FIG. 7 may be executed by the active principle controller (i.e., the controller that is controlling the table 10) and thus either the primary controller 102 or the secondary controller 150 may execute the method of FIG. 7. Further, if the method is executed by the primary controller 102, then the primary controller may determine if the secondary controller 150 is active (since the secondary controller is an agent on the network).

Beginning at step 462, a counter N is initialized (e.g., set to “1”), and at step 464 the active controller transmits a message to each agent on the actuator bus. At step 466 the active controller checks to see if a response is received from controller “N”. If a response is not received, the active controller moves to step 468 to flag controller “N” as inactive and then then to step 472 to inhibit any motion related to controller N. The method moves to step 474 as discussed below. Moving back to step 466, if a response is received, then the active controller moves to step 470 to flag controller N as active and the method moves to step 474.

At step 474, the active controller increments the counter N and at step 476 the active controller checks the value of counter N relative to a maximum value. If the counter N is less than the maximum value, then all agents on the network have not been checked and therefore the method moves back to step 466 and repeats. However, if the counter N is at the maximum value, then all controllers have been checked and the method moves to step 478 where a status report for the agent controllers is generated. Such status report may be used to flag personnel that the table 10 requires maintenance/repair. The method then moves back to step 462 and repeats.

The surgical table in accordance with the invention is advantageous in that it results in reduced wiring, which leads to reduced cost and improved reliability of wiring. It further leads to improved table functionality under single-fault conditions as well as early detection and indication to user of potential failures.

Although the invention has been shown and described with respect to a certain embodiment or embodiments, equivalent alterations and modifications may occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein exemplary embodiment or embodiments of the invention. In addition, while a particular feature of the invention may have been described above with respect to only one or more of several embodiments, such feature may be combined with one or more other features of the other embodiments, as may be desired and advantageous for any given or particular application.

1. A surgical table for supporting a patient during a medical procedure, comprising:
a main support;
a seat section for supporting a lower torso and pelvis of the patient, the seat section connected to the main support;
a back section for supporting an upper torso of the patient, the back section connected to the seat section, wherein the back section is movable relative to the seat section;
at least one electrically-operated actuator coupled to the seat section and the back section, the at least one electrically-operated actuator operative to cause relative movement between the seat section and the back section;
a primary controller communicatively coupled to the at least one electrically-operated actuator; and
a secondary controller communicatively coupled to the at least one electrically-operated actuator, wherein when the primary controller is in a first state the primary controller is configured to control the at least one electrically-operated actuator and the secondary controller is configured to monitor the primary controller, and when the primary controller is in a second state the secondary controller is configured to control the at least one electrically-operated actuator.

2. The surgical table according to claim 1, further comprising a leg section for supporting legs of the patient, the leg section connected to the seat section, wherein the leg section is movable relative to the seat section.

3. The surgical table according to claim 2, wherein the at least one electrically-operated actuator comprises a plurality of electrically-operated actuators, wherein at least one of the plurality of electrically-operated actuators is coupled to the seat section and the leg section, the at least one of the plurality of electrically-operated actuators communicatively coupled to the primary controller and the secondary controller, wherein the primary controller is configured to control the at least one of the plurality of electrically-operated actuators when in the first state, and the secondary controller is configured to control the at least one of the plurality of electrically-operated actuators when the primary controller is in the second state.

4. The surgical table according to claim 1, wherein the secondary controller is configured to monitor the primary controller to determine if the primary controller is in the first state or the second state, and upon the secondary controller determining the primary controller is in the second state the secondary controller is configured to take over control of the at least one electrically-operated actuator.

5. The surgical table according to claim 1, further comprising:
a primary actuator bus coupled to the primary controller, the secondary controller, and the at least one electrically-operated actuator; and
a secondary actuator bus coupled to the primary controller, the secondary controller, and the at least one electrically-operated actuator,
wherein when the primary actuator bus is in a first state, communications between the primary controller, the secondary controller and the at least one electrically-operated actuator occur over the primary actuator bus, and when the primary actuator bus is in a second state, communications between the primary controller, the secondary controller and the at least one electrically-operated actuator occur over the secondary actuator bus.

6. The surgical table according to claim 5, wherein the at least one electrically-operated actuator comprises an electric motor and a motor controller operatively coupled to the electric motor.

7. The surgical table according to claim 1, further comprising a first user input device communicatively coupled to the primary controller and the secondary controller, the first user input device configured to receive inputs from the user and communicate the received inputs to the primary controller and the secondary controller.

8. The surgical table according to claim 7, further comprising a command bus, wherein the first user input device is communicatively coupled to the primary controller and the secondary controller via the command bus.

9. The surgical table according to claim 1, further comprising a second user input device communicatively coupled to the secondary controller, the second user input device configured to receive inputs from the user and communicate the received inputs to the secondary controller.

10. The surgical table according to claim 1, further comprising a third user input device communicatively coupled to the secondary controller, the third user input device configured to receive inputs from the user and communicate the received inputs to the secondary controller.

11. The surgical table according to claim 10, wherein the secondary controller is configured to communicate commands received from the second user input device and the third user input device to the primary controller via the command bus.

12. The surgical table according to claim 1, wherein the at least one electrically-operated actuator comprises a plurality of electrically-operated actuators, further comprising:
a main power source;
a control power switch having a control power input terminal and a plurality of control power output terminals, the control power input terminal coupled to the main power source; and
a motor power switch having a motor power input terminal and a plurality of motor power output terminals, the motor power input terminal coupled to the main power source,
wherein the primary controller and the secondary controller are electrically connected to respective ones of the plurality of control power output terminals, and the plurality of electrically-operated actuators are electrically connected to respective the motor power output terminal.

13. A method for controlling a surgical table configured to support a patient during a medical procedure, the surgical table including a main support, a seat section for supporting a lower torso and pelvis of the patient, the seat section connected to the main support, and a back section for supporting an upper torso of the patient, the back section connected to the seat section, where the back section is movable relative to the seat section, the method comprising:
controlling at least one electrically-operated actuator with a primary controller, the at least one electrically-operated actuator configured to cause relative movement between the seat section and the back section;
monitoring, by a secondary controller, an operational status of the primary controller; and
upon the secondary controller determining the primary controller is malfunctioning, the secondary controller taking over control of the at least one actuator from the primary controller.

14. The method according to claim 13, wherein monitoring by the secondary controller comprises:
the secondary controller querying the primary controller; and
the secondary controller taking over control of the at least one actuator upon the primary controller failing to respond to the query.

15. The method according to claims 13-14, wherein the surgical table further comprises a primary actuator bus coupled to the primary controller, the secondary controller, and the at least one electrically-operated actuator, and a secondary actuator bus coupled to the primary controller, the secondary controller, and the at least one electrically-operated actuator, the method further comprising:
determining a status of the primary actuator bus;
using the primary actuator bus to communicate between the primary controller, the secondary controller and the at least one electrically-operated actuator when the primary actuator bus is in a first state; and
using the secondary actuator bus to communicate between the primary controller, the secondary controller and the at least one electrically-operated actuator when the primary actuator bus is in a second state.

16. The method according to any one of claims 133-15, wherein the surgical table further comprises a first user input device communicatively coupled to the primary controller and the secondary controller via a command bus, the method further comprising:
the secondary controller monitoring a status of the command bus; and
the secondary controller taking over control from the primary controller of the at least one actuator upon the command bus being in a malfunctioning state.

17. The method according to claim 13, further comprising using a first user input device communicatively coupled to the primary controller and the secondary controller to receive inputs from the user and communicate the received inputs to the primary controller and the secondary controller.

18. The surgical table according to claim 17, wherein using the first user interface includes communicating to the primary and secondary controllers over a command bus communicatively coupled to the primary controller and the secondary controller via the command bus.

19. The method according to claim 17, further comprising using a second user input device to receive inputs from the user and communicate the received inputs to the secondary controller.

20. The method according to claim 19, further comprising using a third user input device to receive inputs from the user and communicate the received inputs to the secondary controller.

21. (canceled)
